(19) United States
(12) Patent Application Publication
(10) Pub. No.: US 2025/0261891 A1
(43) Pub. Date: Aug. 21, 2025

(54) ELECTRODE

(71) Applicant: Leonh. Lang, Innsbruck (AT)

(72) Inventor: Thomas KUPSA, Innsbruck (AT)

(21) Appl. No.: 19/203,864

(22) Filed: May 9, 2025

Related U.S. Application Data
(62) Division of application No. 17/494,282, filed on Oct. 5, 2021.
Application No. 17/494,282 is a continuation of application No. PCT/AT2020/060136, filed on Apr. 2, 2020.

(30) Foreign Application Priority Data
Apr. 16, 2019 (AT) A 50343/2019

(51) Int. Cl. A61B 5/259

(52) U.S. Cl. CPC A61B 5/259; A61B 2562/0215; A61B 2562/125; A61B 2562/227

(57) ABSTRACT

An electrode for applying to human skin, has an electrically non-conductive support. The support has on its upper side a projecting, electrically conductive connection element with a connection location for the releasable connection of a signal conductor. A conductor is provided, and the conductor is arranged at least to some extent on the opposite, underside of the support and is connected electrically to the connection element and to a contact medium, which is directed towards the skin. The connection element has at least one protrusion, which extends through the support and, at its end, has a widened region formed by deformation. This deformed, widened region makes it possible to establish, on the one hand, an electrical connection between the conductor and the connection element and, on the other hand, a mechanical fastening of the connection element on the support.

### CROSS REFERENCE TO RELATED APPLICATIONS

The present application is a divisional application of U.S. patent application Ser. No. 17/494,282 filed on Oct. 5, 2021, which is a continuation of International Application PCT/AT2020/060136 filed on Apr. 2, 2020, which claims the benefit of priority to Austrian Application No. A 50343/2019 filed on Apr. 16, 2019, the entire disclosures of which are incorporated herein by reference.

### BACKGROUND OF THE INVENTION

The invention concerns an electrode and a method of producing an electrode.

Medical skin electrodes of that kind can be used as measurement electrodes which derive electrical signals from the human body. They can however also be used as therapy electrodes to feed currents to the human body. For that purpose, the electrodes are glued on to the skin and on their underside generally have an electrically conducting gel or another electrical contact medium which is galvanically in contact with a connecting element of the electrode. An electrical signal conductor can be connected to that connecting element, by way of which conductor currents can be taken from the electrode or fed to the electrode.

One type of electrode has at the top side facing away from the skin a projecting electrically conducting connecting element with a generally substantially ball head-shaped connecting location to which a neck is connected.

In the previous construction of electrodes of that type, the connecting element has a two-part structure. The upper part (upper knob or stud) serves as a contact and anchor element for commercially usual signal conductors, for example ECG lines. Substantially beneath the carrier, that is to say on the side facing towards the skin, there is a lower knob (eyelet) which serves for the transfer of electrical potentials directly from the gel (contact medium) or for transmission to the gel. In that case, the eyelet is connected both mechanically and electrically to the stud, more specifically generally by riveting of the two parts, in such a way that the carrier material of the electrode is fixedly clamped between a holding region of the stud, that projects laterally like a flange, and a likewise holding region of the eyelet. Such a construction affords on the one hand a good mechanical hold for the connecting element to the carrier of the electrode while on the other hand it makes it possible to make the eyelet from materials which have favorable electrical properties for a signal electrode, for example for that purpose it can be coated with silver, in which case the silver coating can in turn be covered over its entire area or at least in a partial region which is in contact with the gel with a layer of silver/silver chloride (Ag/AgCl). There is also the possibility that the eyelet does not directly contact the gel. In those so-called off-center electrodes, a transverse conductor connects the eyelet to the gel.

The electrodes in accordance with the state of the art, however, are costly—in that respect just minor differences in price are significant in relation to mass-produced articles of that kind.

Patent specification AT 519280 A1 to the present applicant therefore proposes that, instead of a frequently usual two-part structure in which the connecting element comprises two parts which are riveted together (stud and eyelet), there is now a single part as the connecting element, which on the one hand provides the connecting location for releasable connection of a signal conductor and which on the other hand is connected (preferably galvanically) to the electrical transverse conductor.

It will be noted, however, that such a one-part configuration of the connecting element still has some disadvantages.

By way of example, an additional support layer is required to sufficiently fix the connecting element to the carrier. On the one hand, that has an effect on the material requirement for an electrode and also has an effect on the period for producing an electrode.

The material requirement and the period required for producing an electrode are however crucial in terms of the manufacturing costs of such an electrode. It is therefore desirable to minimize the material requirement and the period required for producing an electrode.

Therefore, the object of the invention is to provide an electrode which is improved over the electrode disclosed in patent specification AT 519280 A1—in particular, in regard to manufacturing costs and time—and a method of producing such an electrode.

### SUMMARY OF THE INVENTION

To achieve the above object, a connecting element has at least one projection which extends through the carrier and which at its end has an enlarged region formed by deformation. That deformed enlarged region makes it possible on the one hand to provide an electrical connection between the conductor and the connecting element and on the other hand mechanical fixing of the connecting element to the carrier.

In that way, both the material requirement and also the electrode production time can be reduced.

It is also advantageous that the connecting element is connected to the carrier and/or the conductor both in positively locking and also force-locking relationship by the enlarged region formed by deformation.

Particularly preferably, the connecting element comprises a single part which has the connecting location for releasable connection of a signal line.

The connecting element itself can certainly comprise a plurality of materials, for example nickel-plated brass or a plastic doped with conductive material (in particular carbon fibers).

A particularly preferred configuration of the connecting element is one in which it has such a configuration that the connecting element has a substantially ball-shaped head, an adjoining neck of reduced diameter, a holding region which adjoins the end of the neck and which projects laterally in a flange shape and at least one projection adjoining the holding region.

The projection is passed through an opening in the carrier (preferably without making lateral contact therewith) while the holding region projecting laterally in a flange shape bears against the top side of the carrier. The holding region of enlarged diameter which projects laterally in a flange shape holds the connecting element firmly and securely to the carrier material even under high pressure loadings.

The deformed enlarged region of the projection of the holding element bears against the underside of the carrier, that faces towards the skin, or against the conductor, and thus ensures a good hold for the connecting element on the carrier, even in the event of pressure loadings on the connecting element.

The at least one projection can comprise at least a first segment and at least a second segment. In that case, both segments in a starting position are in a horizontal position or in a vertical position, or at least a first one of the at least two segments is in a horizontal position and at least a second one of the at least two segments is in a vertical position, and the holding region is formed by at least one of the at least two segments.

In this embodiment of the invention, deformation of the deformable region can be effected by simply bending over at least one of the at least two segments.

According to a further embodiment of the invention, the laterally projecting flange-like holding region comprises at least two wing segments. The at least two wing segments have a portion inclined with respect to a horizontal position, and the projection is formed by the at least two wing segments. Preferably, the at least two wing segments are at least portion-wise of a sharp-edged configuration.

The at least two wing segments in that case can be of a congruent or non-congruent configuration. In the latter case, in particular, the inclined portions can have differing lengths.

In such an embodiment, it is possible for the connecting element to be introduced into the carrier or conductor without previously making a through opening through the carrier and the conductor—by a combination of a rotary and pressing movement of the connecting element on the top side of the conductor. At least one of the at least two inclined portions can be changed in shape by simply bending in the direction of the underside of the carrier.

If the wing segment portions are of a sharp-edged configuration that makes it easier to penetrate a carrier and a conductor.

In a further embodiment of the invention, the at least one projection is in the form of a bar which narrows in a direction opposite to the holding region. In that way, it is possible for the connecting element to be introduced into the carrier or conductor without previously making a through opening through the conductor and the carrier. That, therefore, saves on a working step.

High demands are not made on the electrical properties of the connecting element in the case of the subject of the invention. It can therefore comprise inexpensive material, such as a simple metal sheet. More specifically, the connecting element does not need to have any particular electrical properties for only the conductor which is in contact with the electrical contact medium can have those electrical properties which are advantageous in terms of bioelectrodes.

In that respect, the conductor can basically have any desired geometry. In preferred embodiments of the invention, however, the conductor can have the form of a rotationally symmetrical or substantially cuboidal conductor plate. That conductor plate can project at least partially over the deformed enlarged region.

In order to achieve a low level of noise and depolarization in the case of defibrillation in respect of an electrode redox, couples are currently used. They can be oxidized or reduced and, in that case, receive or give off at least one electron. The most widely varying substances are used for such depolarization at the present time. Silver/silver chloride and tin/tin chloride are most frequently used. It will be appreciated, however, that all redox couples which permit depolarization of the electrode are possible for the present invention. In that respect, the redox couples can be actively added or possibly generated in situ by reactions.

As for example silver/silver chloride is a relatively costly substance, it is sufficient if in accordance with a further aspect of the invention the conductor preferably has one side an electrically conducting material which is galvanically joined to the connecting element and to the contact medium.

Further costs can be saved by the measure of providing the conductor, preferably at one side, with an electrically conducting material. More specifically, the actual conductor can use inexpensive materials like for example metal or plastic while a second electrically conducting material like for example silver/silver chloride can be used at the transition region to the electrical contact medium (in particular gel), that is critical for the desirable electrical properties of the bioelectrode. It is sufficient if such material is only locally present in that region.

In particular, the conductor can comprise a plastic film provided with an electrically conducting material.

Overall, the basic concept of the invention is to provide the connecting element for the signal conductor in such a fashion that it is well anchored in the electrode while the electrical properties are a less important consideration and thus inexpensive materials can be employed.

On the other hand, the more expensive materials which are provided for the advantageous electrical signal line can be used only in the electrical critical region at the transition to the electrical contact medium (gel). The conductor performs that function. Stated in quite brief terms, it would be said that the electrically conducting connecting element, apart from the basic property of electrical conduction, is primarily responsible for the “mechanics”. The reverse applies in respect of the conductor: it does not need to fulfill any particular mechanical properties and it is only in the region of the transitional location to the electrical contact medium (gel) that it comprises materials which are desirable for that purpose. In that respect, the conductor is responsible for the “electrics” without any particular mechanical functions.

### BRIEF DESCRIPTION OF THE DRAWINGS

Further advantages and details of the invention are described with reference to the drawings, in which:

FIG. 1 is a diagrammatic view from below (later the side facing towards the skin) of the production steps of an embodiment of an electrode according to the invention to the finished electrode,

FIG. 2 is a diagrammatic plan view of the production steps of an embodiment of an electrode according to the invention to the finished electrode, with only a part of the process steps being shown as a plan view,

FIG. 3 shows a sequence of the sections along line A-A in FIG. 1, where the view is to be interpreted as a diagrammatic view for better visualization,

FIG. 4 is a diagrammatic view from below (later the side facing towards the skin) of the production steps of a further embodiment of an electrode according to the invention to the finished electrode,

FIG. 5 is a diagrammatic plan view of the production steps of a further embodiment of an electrode according to the invention to the finished electrode, with only a part of the process steps being shown as a plan view,

FIG. 6 shows a sequence of the sections along line A-A in FIG. 4, where the view is to be interpreted as a diagrammatic view for better visualization,

FIG. 7 is a diagrammatic view from below (later the side facing towards the skin) of the production steps of a further embodiment of an electrode according to the invention to the finished electrode,

FIG. 8 is a diagrammatic plan view of the production steps of a further embodiment of an electrode according to the invention to the finished electrode, with only a part of the process steps being shown as a plan view,

FIG. 9 shows a sequence of the sections along line A-A in FIG. 4, where the view is to be interpreted as a diagrammatic view for better visualization,

FIG. 10 is a diagrammatic view from below of an embodiment of a carrier according to the invention with an adhesive layer,

FIG. 11 is a diagrammatic view from below of a further embodiment of a carrier according to the invention with an adhesive layer,

FIG. 12a is a diagrammatic side view of an embodiment of a connecting element according to the invention,

FIG. 12b is a diagrammatic plan view of an embodiment of a connecting element according to the invention,

FIG. 13a is a diagrammatic side view of an anchoring process of a connecting element according to the invention in a carrier,

FIG. 13b is a diagrammatic plan view (later the side facing away from the skin) of an anchoring process of a connecting element according to the invention in a carrier,

FIG. 14a is a diagrammatic view of a further embodiment of a connecting element according to the invention,

FIG. 14b is a diagrammatic view of a further embodiment of a connecting element according to the invention,

FIG. 15a is a diagrammatic side view of a further embodiment of a connecting element according to the invention,

FIG. 15b is a diagrammatic plan view of a further embodiment of a connecting element according to the invention,

FIG. 16a is a diagrammatic side view of a further embodiment of a connecting element according to the invention,

FIG. 16b is a diagrammatic plan view of a further embodiment of a connecting element according to the invention,

FIG. 17a is a diagrammatic side view of a further embodiment of a connecting element according to the invention,

FIG. 17b is a diagrammatic plan view of a further embodiment of a connecting element according to the invention,

FIG. 18 is a diagrammatic view from below (later the side facing towards the skin) of the production steps of a further embodiment of an electrode according to the invention to the finished electrode,

FIG. 19 is a diagrammatic plan view of the production steps,

FIG. 20 shows a sequence as shown in FIGS. 18 and 19 in a sectional view,

FIGS. 21, 22 and 23 show similar views to FIGS. 18, 19 and 20 but for a further embodiment.

### DETAILED DESCRIPTION OF THE INVENTION

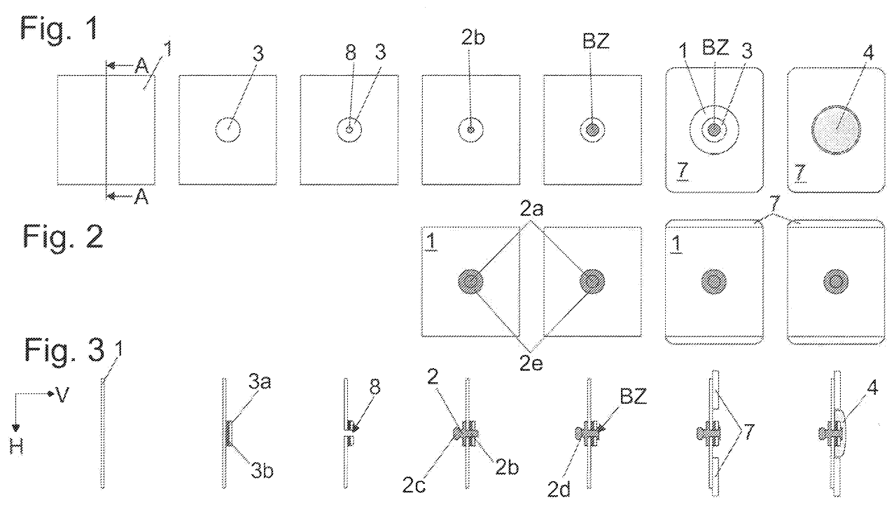

With reference to FIGS. 1 through 3 the procedure of the method for the production of an embodiment of an electrode according to the invention for application to the human skin is now described in greater detail.

The basic starting point is an electrically non-conducting carrier 1. The carrier material serves for anchoring the electrical components of the electrode. It can comprise for example a (flexible) film (for example of PET or TPU) which on the underside facing upwardly in the drawing of FIG. 1 is completely or partially coated with an adhesive which for example can be self-adhesive (pressure sensitive adhesive) or thermoactivatable (hot melt).

Now in a next step a rotationally symmetrical conductor 3 is fixed on that carrier material, in particular by adhesive or by being printed thereon. In accordance with a preferred variant of the invention the conductor has two different electrically conducting materials or an electrically non-conducting material 3b and an electrically conducting material 3a, and the electrically conducting material 3a or one of the two electrically conducting materials is later galvanically connected to the electrical connecting element 2 and to the contact medium 4 (gel).

The illustrated embodiment involves a circular conductor 3 of a plastic film, which is shown in black or gray. The conductor 3 however can also comprise a metal or a conductive plastic doped with carbon fibers.

In the region of the later contact location with the electrical contact medium 4 (gel) that conductor 3 is coated with a layer 3a of for example silver/silver chloride or tin/tin chloride or another redox couple.

In a further step an opening 8 is now provided through the electrical conductor 3 and the carrier 1. That can be done for example by stamping. The connecting element 2 which has a projection 2b which projects beyond the underside of the carrier 1 and the conductor 3 is then introduced.

In the illustrated embodiment, adjoining the substantially ball-shaped head 2c, the connecting element 2 has a neck 2d of reduced diameter, which is adjoined by a holding region 2e projecting laterally in a flange shape, and a projection 2b.

Overall the laterally projecting flange-shaped holding region 2e is of a substantially plate-shaped configuration. It is responsible for distribution and transmission of pressure forces applied to the connecting element 2, to the carrier 1.

When using a connecting element 2 which comprises a single part which has a first end connected to the electrical conductor 3 and which on the other hand has the connecting location 2a at a second end for releasable connection of a signal conductor (not shown here) inexpensive manufacture of the electrode is possible in that way because the generally cost-intensive eyelet (underneath knob) can be omitted. The one-part configuration of the connecting element is sufficient for mechanical anchoring.

The demands made in terms of the electrical properties are low. In that way, it is possible to use simple structures like for example a deep-drawn metal part as the connecting element 2. The somewhat more difficult electrical functions are therefore implemented here not by the otherwise usual eyelet but the conductor 3 which is joined to the electrical contact medium 4 (gel) which is later applied.

This therefore involves separation of the functions. Apart from the basic property of being electrically conducting the electrical connecting element 2 is substantially responsible for the mechanical hold in the electrode while the conductor 3 is substantially freed of mechanical tasks. That makes it possible to adopt a favorable material. In particular, it is possible to provide more costly materials-which are favorable from the electrical point of view-only where (location 3a) contact with the gel later occurs.

As already mentioned the electrically conducting connecting element 2 can comprise a deep-drawn metal sheet. It is then at least partially hollow in its interior. It can however also comprise a conductive plastic, for example ABS, which is doped with conductive carbon fibers.

More desirably the connecting element is of a substantially rotationally symmetrical configuration. Other variants are also possible.

In order to fix the electrical connecting element 2 definitively in the electrode and in particular also to secure it against tensile loadings a next step provides for deforming the projection 2b in such a way as to produce a deformed enlarged region BZ.

Deformation of the projection 2b can be effected in that case by fusing, beading over, spreading or bending over. It is however also possible to use any other suitable method.

The deformation of the projection 2b provides that a galvanic connection is made between the connecting element 2 and the conducting material 3a of the conductor by way of the deformed enlarged region BZ while on the other hand mechanical fixing of the connecting element 2 to the carrier 1 is effected by a positively locking and/or force-locking relationship.

A plaster layer 7 is now applied to the underside of the carrier 1, in particular by adhesive, wherein the plaster layer can preferably be stuck on the skin by means of a patient-side coating of biocompatible plastic in order to fix the electrode.

In that respect it is also possible for the plaster layer to be glued to the carrier 1 by way of a layer applied to the plaster layer and comprising pressure-sensitive adhesive or a thermoactivatable adhesive.

The plaster material ultimately serves to fix the electrode on the patient skin. Suitable plaster materials can comprise for example a film (for example PE), a foam band (for example PE foam) or non-woven materials. The plaster materials are usually coated on the patient side with a biocompatible adhesive.

In the last step in the production of the electrode shown in FIGS. 1 through 3 the electrical contact medium 4 is introduced into a recess provided for same in the plaster material 7. The electrical contact medium 4 permits the (preferably ion-based) conduction of body-generated electrical potentials or device-generated measurement or stimulation currents from the body surface (skin) to the electrical connecting element 2 and vice-versa. The contact medium can for example comprise a gel which is doped with chlorides and which is present either in a more or less liquid form (more or less gelled) or in the form of a cross-linked polymer matrix (hydrogel). It is however also possible to create the electrical contact medium 4 with other means, for example in the form of conductive adhesives or in the form of sponge filled with saline solution.

In any event, the electrical contact medium 4, as the last step in FIGS. 1 through 3 shows, is introduced into the recess in the plaster material 7. It contacts therein the electrically conducting material 3a (in particular silver/silver chloride).

The cooperation of the electrically conducting material 3a, in particular the coating with silver/silver chloride or another suitable material on the one hand and the material of the electrically conducting contact medium 4 on the other hand makes it possible to achieve favorable electrical properties of the electrode like for example noise-free signal transmission or depolarizing effects, in which case the use of the relatively costly electrically conducting material 3a of the conductor 3 can remain restricted to that region in which contact with the contact medium 4 occurs. That further reduces the costs.

Overall in the production shown in FIGS. 1 through 3 there is a “central” electrode in which the connecting element 2 and the contact medium 4 (gel) are arranged directly above each other (i.e., along the longitudinal axis of the connecting element 2).

The method steps which are essential for the embodiment shown in FIGS. 1 through 3 are as follows:
arranging, preferably gluing or printing, a conductor (3) on the underside, (to face towards the skin), of an electrically non-conducting carrier (1),
introducing a connecting element (2) from the top side of the carrier (1) through the carrier (1) in such a way that the projection (2b) of the connecting element (2) projects on the opposite underside of the carrier (1) and the connecting element (2) bears against the top side of the carrier (1)—preferably with a laterally projecting plate-shaped holding region (2e)—, and
deformation of the projection (2b) of the connecting element (2) in such a way as to produce a deformed enlarged region (BZ) which makes an electrically conductive connection between the connecting element (2) and the conductor (3), and also provides for mechanical fixing of the connecting element (2) to the carrier (1).

Finally the following steps are then also implemented to finish the electrode:
applying—preferably gluing—a plaster layer (7) which is adhesive on the skin side to the carrier (1), and
introducing an electrical contact medium (4)—preferably a gel—into the recess in the plaster layer (7) in such a way that the subjacent conductor (3) is contacted.

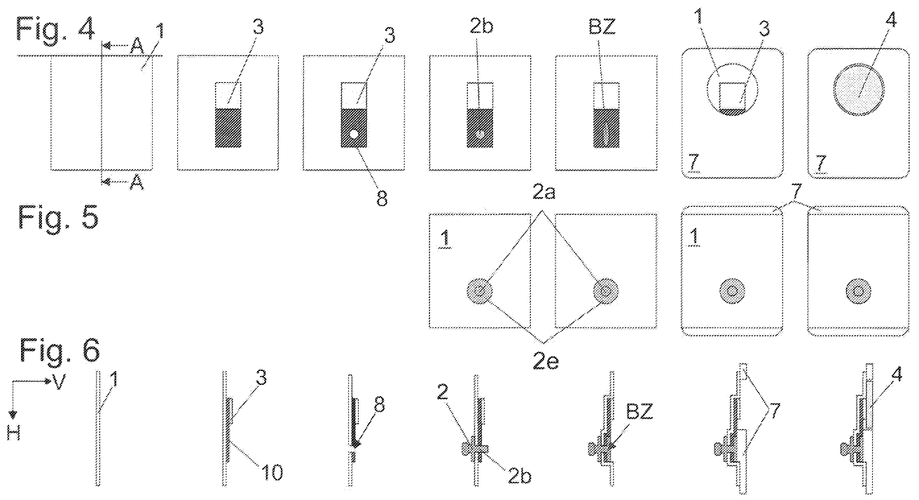

In the embodiment shown in FIGS. 4 through 6 most of the method steps are the same as those in FIGS. 1 through 3, for which reason the same references denote the same parts.

The difference is essentially that there is provided a “decentral” electrode. In other words, the contact medium 4 on the one hand and the connecting element 2 on the other hand are displaced relative to each other in a horizontal plane H.

In such an embodiment, it is necessary to provide an electrically conducting transverse conductor 10 which makes a galvanic connection between the conductor 3 and the connecting element 2.

In deformation of the projection 2b pressure can be exerted on the layers so that they are correspondingly contoured and interconnected. The cross-section shown in FIG. 6, after attachment of the plaster layer 7 with the edges shown therein, is however only to be viewed as a diagrammatic illustration. In actual fact the layer thicknesses are generally smaller and the configurations of the layers are substantially rounded off.

It can further be seen that the deformed enlarged region BZ can also no longer be circular but of a lamellar configuration. The deformed enlarged region BZ can basically be of any desired shape.

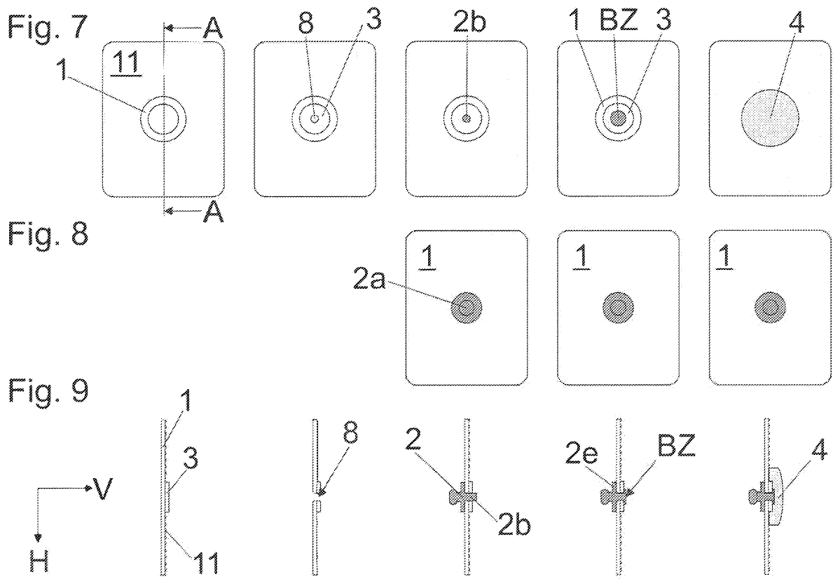

In the embodiment shown in FIGS. 7 through 9 most of the method steps are the same as those in FIGS. 1 through 3, for which reason identical references also denote the same parts.

The difference is substantially that there is provided on the carrier 1 a biocompatible adhesive layer 11 for attaching the electrode to the skin of a patient. The plaster layer 7 can thus be eliminated and a further process step is saved.

In this case the adhesive layer 11 can be applied prior to or after application of the conductor 3 to the carrier 1 or the adhesive layer 11 is already provided on the starting material of the carrier 1.

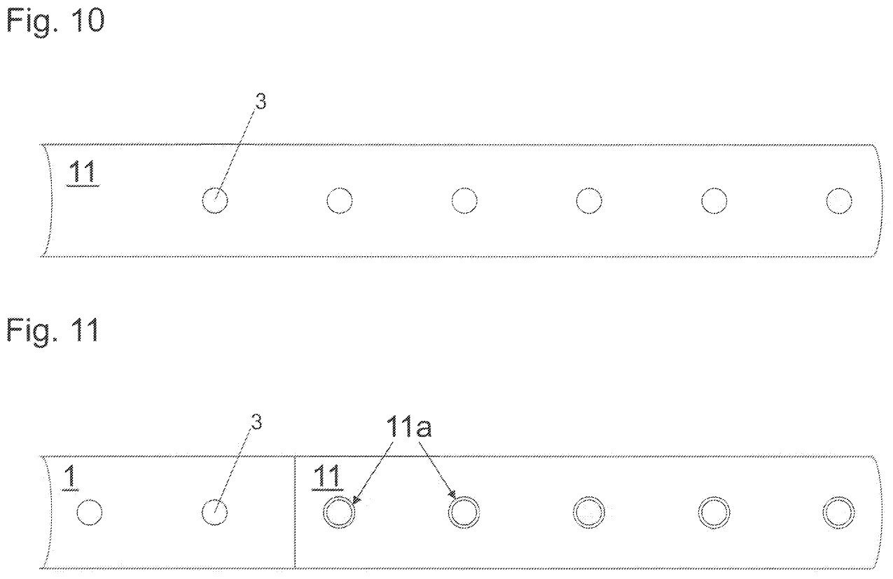

The above-mentioned variants for applying the adhesive 11 are shown in FIGS. 10 and 11.

In FIG. 10 the adhesive 11 is already present on the carrier 1 or is applied prior to application of the conductor 3. The conductor 3 is then applied to the adhesive layer 11. In that case the conductor 3 can be held by the adhesive layer 11 whereby the conductor 3 does not have to be additionally glued to the carrier 1.

In FIG. 11 the conductor 3 is applied to the carrier 1 and then the adhesive 11 is applied to the carrier 1. In this case there is provided an opening 11a so that the conductor 3 is not covered by the adhesive 1.

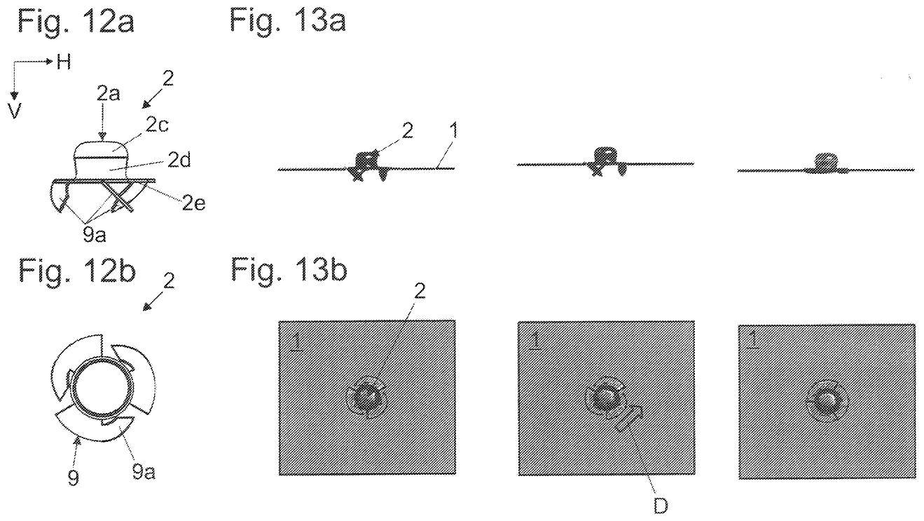

FIGS. 12a and 12b show an embodiment of a connecting element 2 according to the invention. It can be seen that the connecting element 2 has wing segments 9 which form both the projection and also the holding region of the connecting element 2. The wing segment portions 9a which are inclined with respect to a horizontal position H can be of the same or differing lengths. It is also conceivable that the wing segments 9 are of a sharp-edged configuration at least portion-wise to facilitate penetrating a carrier 1 and a conductor 3.

FIGS. 13a and 13b show diagrammatic views of an anchoring procedure for a connecting element according to the invention in a carrier, with a connecting element 2 as shown in FIGS. 12a and 12b.

For that purpose in a first step the connecting element 2 is pushed from a top side of a carrier 1, that later faces away from the skin, through the carrier 1 and the conductor 3 (not shown) which is attached to the underside of the carrier 1. This means that the connecting element 2 penetrates the carrier 1 and the conductor 3 with the wing segment portions 9a.

In a next step the connecting element 2 is rotated in a direction D. That provides for better anchorage of the connecting element 2 in the carrier 1.

In a last step the wing segment portions 9a are bent up in the direction of the underside of the carrier 1 beyond a horizontal position H whereby the carrier 1 and the conductor 3 are clamped. This also ensures an electrical connection of the connecting element 2 to the conductor 3 and a mechanical fixing of the connecting element 2 on the carrier 1. It will be appreciated however that it is also possible for the wing segment portions to be only bent up until they are in a horizontal position H.

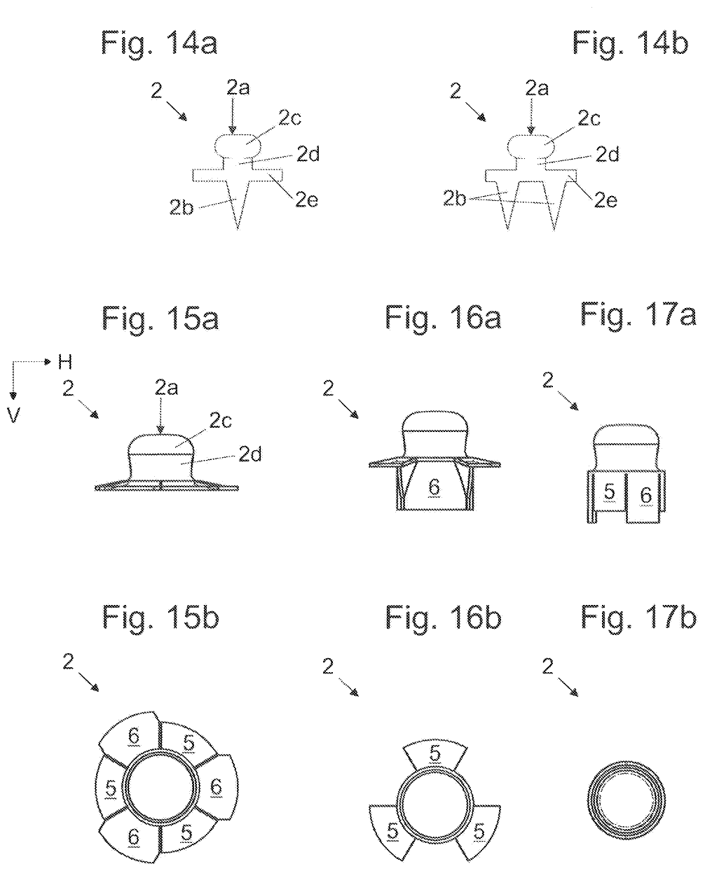

FIG. 14a shows an embodiment of a connecting element 2 in which the projection 2b is in the form of a tapering spike. In that way it is possible to introduce the connecting element 2 into the carrier 1 and the conductor 3 without previously producing a through opening 8 through the conductor 3 and the carrier 1. That therefore saves on a working step.

FIG. 14b shows an embodiment of a connecting element 2 in which there are two projections 2b in the form of tapering spikes. It will be noted however that there can be any number of projections 2b. In addition it is also possible to provide a plurality of projections 2b which are not of a spike shape. In that case the plurality of projections 2b can be arranged on the connecting element 2 in rotationally symmetrical or non-rotationally symmetrical relationship.

FIGS. 15a through 17b show embodiments of a connecting element 2 in which the projection and the flange-like holding region of the connecting element 2 are formed from at least one first segment 5 and at least one second segment 6.

It can also be seen that the second segments 6 are longer than the first segments 5. The segments 5, 6 can also be of equal length or the segments 5 can be longer than the segments 6.

FIG. 15a shows the connecting element in a front view when all segments 5, 6 are in a horizontal position H. FIG. 15b shows the corresponding plan view.

FIG. 16a shows a front view of the connecting element when the segments 5 are in a horizontal position H and the segments 6 are in a vertical position V. FIG. 16b shows the corresponding plan view.

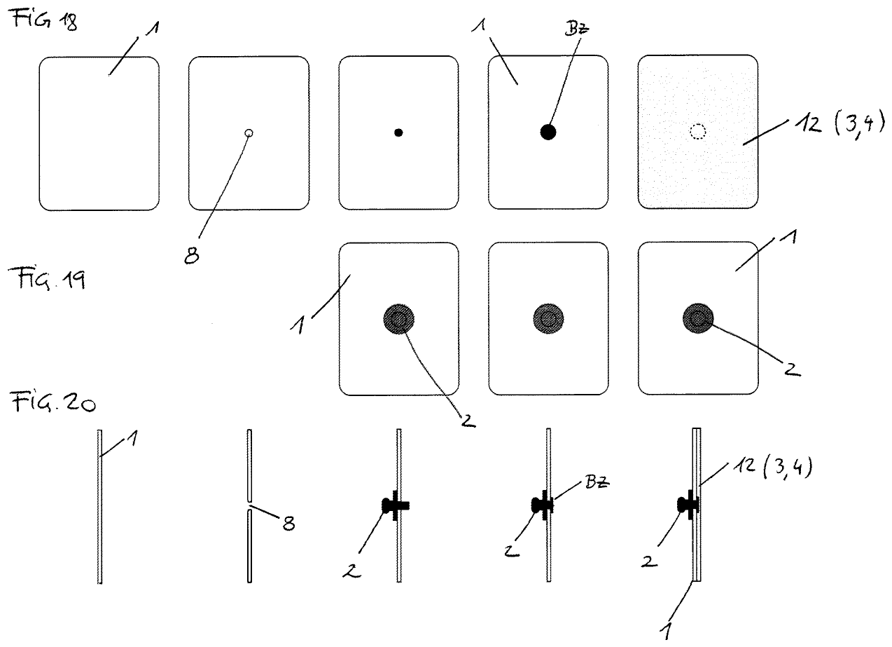

FIG. 17a shows a front view of the connecting element when all segments 5, 6 are in a vertical position V. FIG. 17b shows the corresponding plan view.

In a connecting element 2 as shown in FIGS. 15a and 15b, prior to fitment of the connecting element 2 into the carrier 1, at least a first segment 5 of the at least two segments 5, 6 is moved into a vertical position V and after introduction of the connecting element 2 into the carrier 1 the at least one first segment 5 is moved into a horizontal position H again.

In an embodiment of a connecting element 2 as shown in FIGS. 17a and 17b before the connecting element 2 is fitted into the carrier 1 at least the one first segment 5 is moved into a horizontal position H while after the connecting element 2 is introduced into the carrier 1 at least one second segment 6 is moved into a horizontal position H.

In the previous embodiments shown in FIGS. 1 through 17b the conductor 3 and the contact medium 4 were formed by separate components and also comprise preferably different materials.

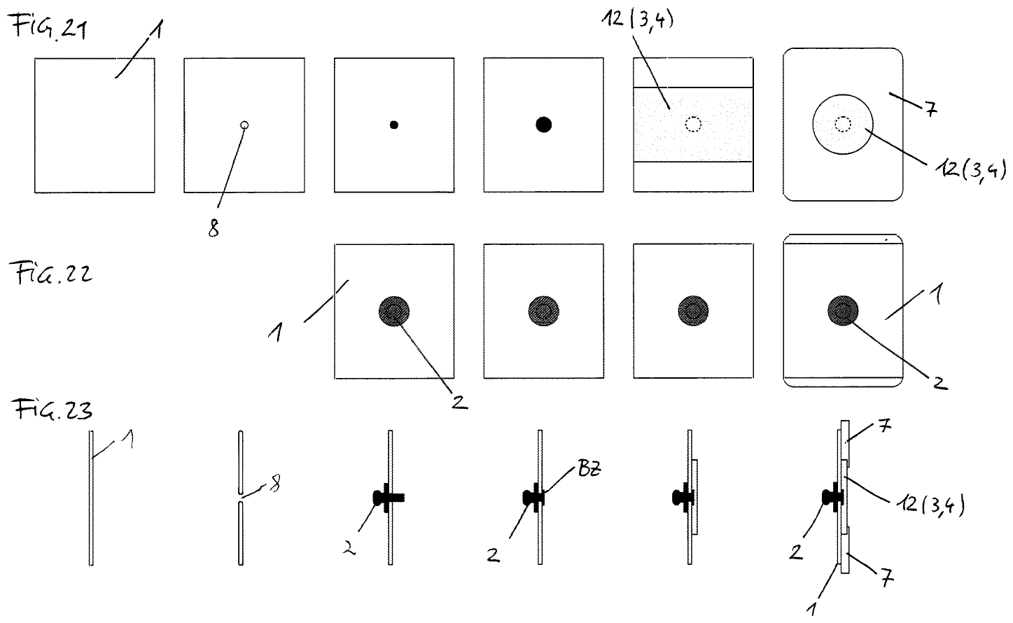

There is however also the possibility of the conductor 3 and the contact medium 4 being formed by one and the same component, preferably by an electrically conducted adhesive, so that it is possible to save overall on components. That is described in greater detail hereinafter with reference to FIGS. 18 through 23.

In the embodiment shown in FIGS. 18 and 20 an opening 18 is first produced in a carrier, a connecting element is inserted into the opening and then the region passing therethrough is enlarged by deformation. The enlarged region BZ then holds the connecting element 2 securely to the carrier. A layer of electrically conductive adhesive 12 is then applied, by means of which the finished electrode can be stuck on the skin of the patient. The electrically conductive layer comprising the adhesive 12 therefore performs the function of the conductor 3 and the contact medium 4.

In the embodiment shown in FIGS. 18 and 20 the adhesive 12 is applied on the underside of the carrier 1 over the full surface area.

In the embodiment shown in FIGS. 21 through 23 most production steps are identical to the example of FIGS. 18 through 20. It is only in the penultimate step that the electrically conductive adhesive 12 which performs the function of the conductor 3 and the contact medium 4 is applied not over the full surface area but only partially—as shown in FIG. 21, in the form of a strip—.

Additionally in a last step an also adhesive plaster material 7 having a central opening is applied, through which the electrically conductive adhesive is still free as the contact medium. Overall therefore adhesive connection to the skin is effected on the one hand by way of the adhesive plaster material 7 and on the other hand by way of the electrically conductive adhesive 12. The advantage of the variant shown in FIGS. 21 through 23 is excellent adhesion by the adhesive plaster material 7 and a saving in terms of electrically conductive adhesive in comparison with the variant involving the full surface area as shown in FIGS. 18 through 20. In return in respect of the last-mentioned Figures, it is possible to dispense with an additional component, namely the plaster material 7.

In the embodiments of the invention it is possible in particular for a second layer of the conductor—as described above—to be formed by a layer of silver/silver chloride or tin/tin chloride or another redox couple.

It is however also possible that in addition other electrically conducting components are provided with such redox couples, in particular also the contact medium 4 and/or the connecting element 2. In the sense of economically managing with the relatively costly redox components not all conductive elements will be simultaneously provided with such redox couples, although that is also theoretically possible. As already mentioned, it is sufficient if a second layer of the conductor 3 is provided with such a redox couple. In principle it is also possible to save on the redox couples entirely and to provide that neither the connecting element 2 nor the conductor 3 nor the contact medium 4 contains such a redox couple.

### LIST OF REFERENCES

1 carrier
2 connecting element
2a connecting location
2b projection
2c head
2d neck
2e holding region
2f lower holding region
3 conductor
3a electrically conducting material
3b electrically non-conducting material
4 contact medium
5 first segment
6 second segment
7 plaster layer
8 opening
9 wing segment
9a wing segment portion
10 transverse conductor
11 adhesive layer (skin adhesive)
11a opening
12 electrically conductive adhesive
H horizontal
V vertical
BZ deformed enlarged region

1. A method of producing an electrode for application to the human skin, the electrode including an electrically non-conducting carrier having a top side to face away from the skin, a projecting electrically conducting connecting element having a connecting location for releasable connection of a signal conductor, and a conductor at least partially on a bottom side of the carrier opposite the top side, the conductor being a discrete member separate from, and electrically connected to, the connecting element and to a contact medium arranged to face towards the skin, and the connecting element having a projection passing through the carrier and having an end with an enlarged deformed region such that the end forms an electrical connection between the conductor and the connecting element and also provides a mechanical fixing of the connecting element to the carrier via the deformed enlarged region, the method comprising:
arranging the conductor on an underside of the electrically non-conducting carrier so as to face the skin,
introducing the connecting element from a top side of the carrier through the carrier such that the projection of the connecting element projects on the opposite underside of the carrier and the connecting element bears against the top side of the carrier with a laterally projecting plate-shaped holding region, and
deforming the projection of the connecting element to produce the deformed enlarged region to form the electrically conductive connection between the connecting element and the conductor and provide mechanical fixing of the connecting element to the carrier.

2. The method as set forth in claim 1, further comprising, prior to introducing the connecting element, forming a through opening through the carrier and the conductor.

3. The method as set forth in claim 1, further comprising forming a through opening by penetrating the carrier and the conductor by the projection of the connecting element from an upper side of the carrier remote from the skin.

4. The method as set forth in claim 1, wherein the introducing of the connecting element comprises rotating the connecting element from the top side of the carrier into the carrier and the conducting plate so that wing segments of the connecting element pass through the carrier and the conducting plate.

5. The method as set forth in claim 1, wherein the deforming of the projection of the connecting element includes:
fusing of the projection, and/or
beading over of the projection, and/or
spreading the projection open, and/or
bending the projection over.

6. The method as set forth in claim 1, further comprising:
applying an adhesive plaster layer on the skin side to the carrier; and
introducing an electrical contact gel medium into a recess in a plaster layer on the skin side of the carrier such that the subjacent conductor is contacted.

7. The method as set forth in claim 1, further comprising, prior to introducing the connecting element, coating the carrier over a full surface area or a part of a surface area with a skin adhesive.

8. The method as set forth in claim 7, further comprising, after coating of the carrier with the skin adhesive, applying an electrical contact medium such that the subjacent conductor is contacted.
